(19) United States
(12) Patent Application Publication
(10) Pub. No.: US 2006/0241670 A1
(43) Pub. Date: Oct. 26, 2006

(54) Series of closed operation accupotome

(75) Inventor: Hanzhang Zhu, Beijing (CN)

Correspondence Address: HAMRE, SCHUMANN, MUELLER & LARSON, P.C., P.O. BOX 2902, MINNEAPOLIS, MN, 55402-0902, US

(73) Assignees: Hanzhang ZHU, Beijing (CN); Feng JIN, AnQing ShiHau (CN)

(21) Appl. No.: 11/392,390

(22) Filed: Mar. 29, 2006

(30) Foreign Application Priority Data
Mar. 30, 2005 (CN) 200510059633.0

(51) Int. Cl. A61B 17/34

(52) U.S. Cl. 606/189

(57) ABSTRACT

The present invention relates to a series of closed operation accupotome, including 12 types of accupotome with 33 models, each type of accupotome having an accupotome handle, an accupotome body and an accupotome head, which are connected in this order. It is characterized in that the handle, the body or the head may have various shapes depending on various diseases, and that the flat surface of the handle and the flat wedged surface of the head are in the same plane. The advantages of this invention are: entering into human body without cutting skin, to make the open surgery treatment turn into a closed surgery, and to make chemical therapy turn into physical therapy. Thus, it avoids sequelae and complications brought by the open surgery, and adverse reactions caused by the chemical medications. In addition, it improves curative effects, shortens course of diseases, simplifies therapeutic process, and saves a lot of medical expenses.

### FIELD OF INVENTION

This invention relates to a series of medical instruments for special purpose used in closed operation under the guidance of basic theories of accupotomology. In particular, it relates to a series of closed operation accupotome.

### BACKGROUND OF THE INVENTION

Currently, clinical treatments for various diseases mainly are medication, acupuncture, and surgery, etc. Medication and acupuncture have certain effects for some functional diseases. However, medication may have side effects to the human body; while acupuncture may not have adverse effects, it can only perform lifting & thrusting and twirling & rotating manipulation, but cannot perform dissection and cutting. For diseases such as chronic soft tissue injury, malunion, hyperosteogeny, only surgery can be applied. However, surgery is complicated, has long incision, may seriously injure normal tissue, bring a lot of pain to the patients, and cause high cost. In addition, surgery may cause sequelae and complications at different levels, and sometimes may lead to lifetime handicapped, or even bring the life in danger.

### SUMMARY OF THE INVENTION

The object of the present invention is to provide a series of closed operation accupotome. Such accupotome can be inserted into human body without cutting the skin, which integrates acupuncture and moxibustion with scalper, to overcome the abuses of the current treatments. It is a closed operation instrument that further simplifies the treatment process, effectively avoids or decreases sequelae, complications from the surgery and the adverse reaction from the chemical drugs, and improves curative effects.

A technical solution of the present invention: Such series of closed operation accupotome includes type I flat neat accupotome, type II osteotome accupotome, type III oblique neat accupotome, type IV circular edge accupotome, type V concave edge accupotome, type VI sword edge accupotome, type VII injection accupotome, type VIII bill edge accupotome, type IX scissor edge accupotome, type X revolving edge accupotome, type XI probe form accupotome, and type XII curved form accupotome, all together 12 types with 33 models. Each type of accupotome has an accupotome handle, an accupotome body and an accupotome head, connecting in that order. The features lie on that different types of accupotome have different shapes of the handle, the body or the head, same type but different model accupotome have different length or diameter of the accupotome body, to accommodate treatment for various diseases and various parts and depth of the same disease.

The handle of the above accupotome: The accupotome handle of type II, type VII and type X is in flat trapezium shape, the accupotome handle of other types is in flat calabash shape, the accupotome handle of type VII has a jack that connects to an injector, the accupotome handle of type IX has two wires that connects with scissor edge of the head, and the accupotome handle of type X has a wire that connects with three-piece micro loose-leaf blade of the head.

The body of the above accupotome: Type XI is in flat slip shape with a blade at one side, type XII has a cross section of 45° degree angle sector with a blade at the acute angle, and other types is column shaped in which type VII, IX and X has a thin bore therein.

The head of the above accupotome: Type X is a three-piece micro loose-leaf blade, type XI is probe form, type XII is coniform, and other types is wedge form with various shaped blade at the tips.

The flat surface of the handle and the flat wedge surface of the head are in the same plane, with one direction.

The handle of the above accupotome is made of 2Col3 material, and the body and head are made of 4Col3 material.

The method of using the accupotome of the present invention is:
1. Fixed-point: After confirming pathological changes part and its anatomy, determining the best inserting point. Marking the inserting point with gentian violet, performing routine disinfection, and then covering a sterile drape on the skin with the inserting point being exposed.
2. Fixed-direction: To make the cutting edge in parallel with the blood vessel, nerves and muscular fibers, and press the edge on the inserting point. Then, determine the acupotome body and angle of inserting surface according to the path.
3. Pressurization & separation: After performing step 2), hold the acupotome handle with thumb and forefinger of right hand, with other three fingers to hold the acupotome body. Giving a pressure to the acupotome but not inserting the skin, so as to form a long notch at the inserting point. The edge is in parallel direction with important blood vessel, nerves and muscular fibers. Thus, nerve and blood vessel can be separated at each side of the edge.
4. Insertion: With continued pressure, upon feeling firmness, the skin under the edge is pushed close to the bone surface. With a little more pressure, the acupotome inserts into the skin. By then, the notch at the inserting point essentially disappeared, nerve and blood vessel expand at each side of the acupotome body. Acupotome operation can be performed according to needs.
5. After inserting into human body, according to various diseases and the depth of the pathological changes part, choose different type different model acupotome to perform different manipulates. The manipulates include dissecting, cutting, promoting the blood, etc.

The advantages of the present invention include: Comparing with prior art, the series of closed operation acupotome integrates Chinese science of acupuncture and surgery scalpel. Such acupotome can insert into human body without cutting the skin, which integrates acupuncture and moxibustion with scalper, to replace open surgery with closed surgery, and to change chemical therapy into physical therapy, so as to avoid sequelae and complications from open surgery and the adverse reactions from chemical drugs, and to improve curative effects, shorten disease course, simplify therapeutic process and save a large amount of medical expenses. The sample survey result of the effect and expenses of the acupotome done by Academy of Mathematics and Systems Science, Chinese Academy of Sciences show: For diseases that can be treated by acupotome, the cure rate of acupotome is 1.26 times of that of surgery, and is 4.15 times of that of drug. Every year it saved RMB60.564 billion yuan comparing with surgery expenses, and saved RMB17.224 billion yuan comparing with drugs and other therapies. The series of acupotome is suitable for chronic soft tissue injury, orthopaedics, part of internal medicine, surgery, pediatry, gynaecology, otolaryngology, dermatology, etc.

### BRIEF DESCRIPTION OF THE DRAWINGS

FIG. 1 is a front view of type I flat neat acupotome.

FIG. 2 is a side view of type I flat neat acupotome.

FIG. 3 is a sectional enlarged front view of a head of type I flat neat acupotome.

FIG. 4 is a sectional enlarged side view of a head of type I flat neat acupotome.

FIG. 5 is a front view of type II osteotome acupotome.

FIG. 6 is a sectional enlarged front view of a head of type II osteotome acupotome.

FIG. 7 is a front view of type III oblique neat acupotome.

FIG. 8 is a front view of type IV circular edge acupotome.

FIG. 9 is a front view of type V concave edge acupotome.

FIG. 10 is a front view of type VI sword edge acupotome.

FIG. 11 is a front view of type VII injection acupotome.

FIG. 12 is a front view of type VIII bill edge acupotome.

FIG. 13 is a front view of a head of type IX scissor edge acupotome, when the scissor edge is in open position.

FIG. 14 is a front view of type IX scissor edge acupotome.

FIG. 15 is a front view of type X revolving edge acupotome.

FIG. 16 is a partial view of type X revolving edge acupotome, when the blades are in close position.

FIG. 17 is a partial view of type X revolving edge acupotome, when the blades are in open position.

FIG. 18 is a side view of type X revolving edge acupotome, when the blades are in open position.

FIG. 19 is a front view of type XI probe form acupotome.

FIG. 20 is a sectional enlarged front view of a head of type XII curved form acupotome.

FIG. 21 is a front view of type XII curved form acupotome.

### DETAILED DESCRIPTION OF THE PREFERRED EMBODIMENT

“Open operation” of this invention refers to cutting the skin by use of surgery scalpel under aseptic condition in the operating room, to cut and dissect the body tissue under orthoptic condition.

“Closed operation” refers to using special medical instruments—series of closed operation acupotome, inserting into human body without cutting the skin, to function as surgery scalpel to perform cutting and dissecting. That is, to perform operation similar to surgery under closed condition.

“Structure model” refers to the shape of the acupotome handle, acupotome body and acupotome head, size of acupotome handle and acupotome head, and diameter of acupotome body.

(1) Type I Flat Neat Acupotome

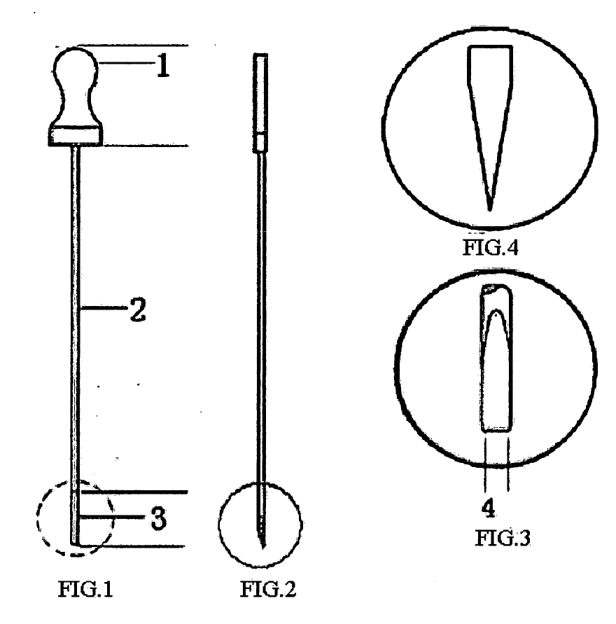

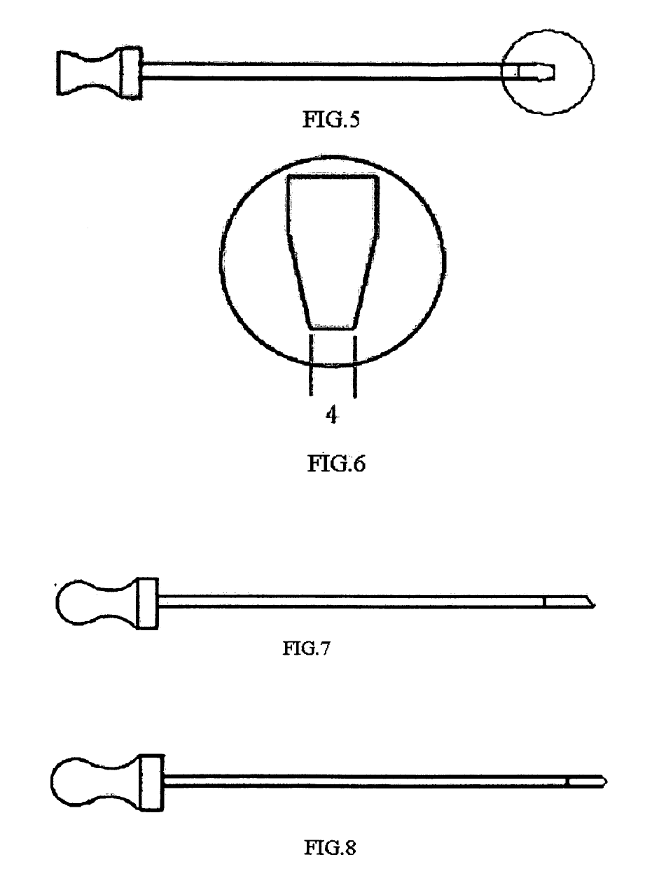

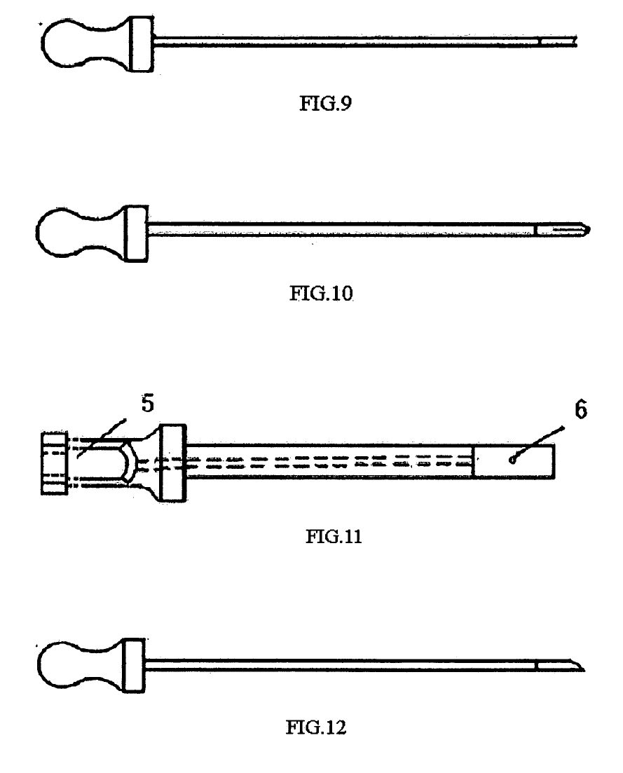

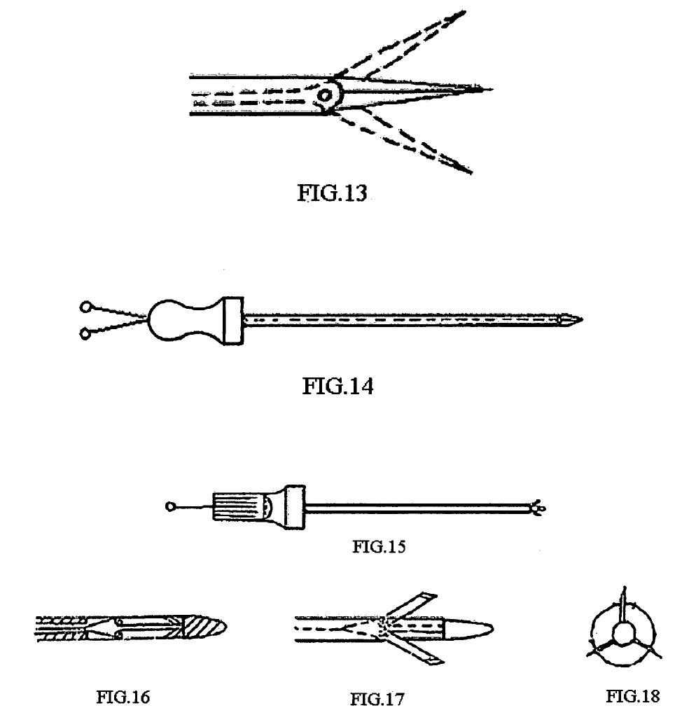

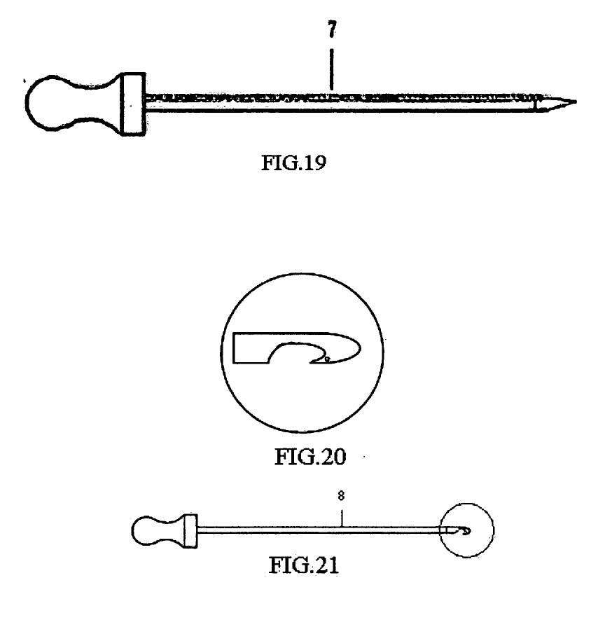

Reference numerals 1, 2, 3 of FIG. 1 and numeral 4 of FIG. 3 refer to acupotome handle, acupotome body, acupotome head, and edge of the acupotome head.

According to the size, type I acupotome has seven models, i.e., type I model 1, type I model 2, type I model 3, type I model 4, type I model 5, type I model 6, and type I model 7.

Type I model 1 acupotome is 15 cm in length, in which the handle 1 is 2 cm long, the body 2 is 12 cm long, and the head 3 is 1 cm long. The acupotome handle is flat calabash shaped. The acupotome body is cylinder form with a diameter of 1 mm. The acupotome head is wedge form, with 15° degree angle at the tip in the side view. Two sides of the angle and the body line are smooth curves. Same below, the end is flat with edge, which has a cutting edge of 0.8 mm. The cutting edge is flat neat that the edge line is perpendicular to the body line. The edge line and the handle are within the same plane. Only within the same plane can distinguish the direction of the edge line in the body from the direction of the handle after the needle inserts into the muscle.

Type I model 2 acupotome has same structure as type I model 1, wherein the acupotome body is 9 cm long. Type I model 3 acupotome has same structure as type I model 1, wherein the acupotome body is 7 cm. Type I model 4 acupotome has same structure as type I model 1, wherein the acupotome body is 4 cm.

Type I model 5 acupotome has a total length of 10 cm, in which the handle is 2 cm long, the body is 7 cm long, and the head is 1 cm long. The handle, body and head have same shape as type I model 5, wherein the body diameter is 0.5 mm, and the edge line is 0.4 mm. Type I model 6 acupotome has same structure as type I model 5, in which the body is 4 cm long. Type I model 7 acupotome has same structure as type I model 5, in which the body is 2 cm long.

Type I model 1, type I model 2, type I model 3, type I model 4 are used for treating various soft tissue injuries, joint injuries and other diseases. Type I model 5, type I model 6 and type I model 7 are used for treating cornea and other mucosa surface.

(2) Type II Osteotome Acupotome

Reference numeral 4 is the edge line of the acupotome head. According to the size, it has two models, i.e., type II model 1 and type II model 2.

Type II model 1 acupotome has a total length of 12.5 cm, in which the handle is 2.5 cm long, the body is 9 cm long, and the head is 1 cm long. The handle is in flat trapezium shape. The body is cylinder form with a diameter of 3 mm. The head is wedge form, the end of which is flat with edge. The cutting edge line at the end is 0.8 mm. The edge line and the handle are in the same plane, and the edge is flat neat.

Type II model 2 acupotome has same structure as type II model 1, in which the body is 11 cm long.

Type II model 1 is used for small malunion osteoclasia and joint fusion decollement. Type II model 2 is used for big malunion osteoclasia and joint fusion decollement.

(3) Type III Oblique Neat Acupotome

According to size, it has three models, i.e., type III model 1, type III model 2, and type III model 3.

Type III model 1 acupotome has a total length of 15 cm, in which the handle is 2 cm long, the body is 12 cm long, and the head is 1 cm long. The handle is flat calabash shaped. The body is cylinder form with a diameter of 1 mm. The head is wedge form, the end of which is flat with edge. The edge line is 0.8 mm. The edge is oblique neat, the edge line and the body line form an obtuse angle. Also, the edge line and the handle are within the same plane. Only within the same plane can distinguish the direction of the edge line in the body from the direction of the handle after the needle inserts into the muscle.

Type III model 2 acupotome has same structure as type III model 1, wherein the body is 9 cm long. Type III model 3 acupotome has same structure as type III model 1, wherein the body is 7 cm long.

The acupotome is used for fascia, periost, and dermotomy in the skin.

(4) Type IV Circular Edge Acupotome

According to the size, it has three models, i.e., type IV model 1, type IV model 2, and type IV model 3.

Type IV model 1 acupotome has a total length of 15 cm, in which the handle is 2 cm long, the body is 12 cm long, and the head is 1 cm long. The handle is flat calabash shaped. The body is cylinder form with a diameter of 1 mm. The head is wedge form, the end of which is flat with edge. The edge line is 0.8 mm. The edge is convex arc deviated from the acupotome handle. The edge line and the handle are within the same plane. Only within the same plane can distinguish the direction of the edge line in the body from the direction of the handle after the needle inserts into the muscle.

Type IV model 2 acupotome has same structure as type IV model 1, wherein the body is 9 cm long. Type IV model 3 acupotome has same structure as typee IV model 1, wherein the body is 7 cm long.

This acupotome is used for flicking nerves, and dissecting periost, fascia and necrotic tissue.

(5) Type V Concave Edge Acupotome

According to the size, it has three models, i.e., type V model 1, type V model 2, and type V model 3.

Type V model 1 acupotome has a total length of 15 cm, in which the handle is 2 cm, the body is 12 cm, and the head is 1 cm. The handle is flat calabash shaped. The body is cylinder form with a diameter of 1 mm. The head is wedge form, the end of which is flat with edge. The edge line is 0.8 mm. The edge is concave arc towards the handle. The edge line and the handle are within the same plane. Only within the same plane can distinguish the direction of the edge line in the body from the direction of the handle after the needle inserts into the muscle.

Type V model 2 acupotome has same structure as type V model 1, wherein the body is 9 cm long. Type V model 3 acupotome has same structure as type V model 1, wherein the body is 7 cm long.

This acupotome is used for cutting the contracture fascia around the tiny nerves.

(6) Type VI Sword Edge Acupotome

According to the size, it has three models, i.e., type VI model 1, type VI model 2, and type VI model 3.

Type VI model 1 acupotome has a total length of 15 cm, in which the handle is 2 cm long, the body is 12 cm long, and the head is 1 cm long. The handle is flat calabash shaped. The body is cylinder form with a diameter of 1 mm. The head is wedge form, the end of which is flat with edge. The edge line is 0.8 mm. The edge is sword neat, and its tip forms 60° degree angle. Both lines of the angle have blades, and the angle lines and the body line form an obtuse angle. The edge line and the handle are within the same plane. Only with the same plane can distinguish the direction of the edge line in the body from the direction of the handle after the needle inserts into the muscle.

Type VI model 2 acupotome has same structure as type VI model 1, wherein the body is 9 cm long. Type VI model 3 acupotome has same structure as type VI model 1, wherein the body is 7 cm.

This acupotome is used for dissecting the cut mark in the muscle, fascia and tendinis.

(7) Type VII injection acupotome

According to the size, it has three models, i.e., type VII model 1, type VII model 2, and type VII model 3.

Type VII model 1 acupotome has a total length of 15 cm, in which the handle is 2 cm long, the body is 12 cm long, and the head is 1 cm long. The handle is flat trapezium shaped, with a jack 5 connecting to the injector. The body is cylinder form with a diameter of 1 mm. The body has a thin bore therein, one end connecting to the injector jack, and the other end connecting to the bore 6 at the edge. The head is wedge form, the end of which is flat with edge. The edge line is 0.8 mm. The bore 6 is 0.2 cm away from the edge tip, and communicates with the injector jack. The edge line and the handle are within the same plane. Only within the same plane can distinguish the direction of the edge line in the human body from the direction of the handle after the needle inserts into the muscle.

Type VII model 2 acupotome has same structure as type VII model 1, wherein the body is 9 cm long. Type VII model 3 acupotome has same structure as type VII model 1, wherein the body is 7 cm long.

This acupotome is used for diseases that need to dissolute large area and that need partial drug injection.

(8) Type VIII Bill Edge Acupotome

According to the size, it has three models, i.e., type VIII model 1, type VIII model 2, and type VIII model 3.

Type VIII model 1 acupotome has a total length of 15 cm, in which the handle is 2 cm long, the body is 12 cm long, and the head is 1 cm long. The handle is flat calabash shaped. The body is cylinder form with a diameter of 1 mm. The head is wedge form, the end of which is flat with edge. The edge line is 0.8 mm. The edge line and the body line form smooth curve, such as beak shape. The edge line and the handle are within the same plane. Only within the same plane can distinguish the direction of the edge line in the human body from the direction of the handle after the needle inserts into the muscle.

Type VIII model 2 acupotome has same structure as type VIII model 1, wherein the body is 9 cm long. Type VIII model 3 acupotome has same structure as type VIII model 1, wherein the bodyd is 7 cm long.

This acupotome is used for dissecting linked tissue surface and cutting internal cystid foci.

(9) Type IX Scissor Edge Acupotome

According to the size, it has three models, i.e., type IX model 1, type IX model 2, and type IX model 3.

Type IX model 1 acupotome has a total length of 14.5 cm, in which the handle is 2 cm long, the body is 12 cm long, and the head is 0.5 cm long. The handle is flat calabash shaped. The body is cylinder form with a diameter of 1.2 mm, which has a thin bore therein. The head is wedge form, the end of which is flat with edge. The edge line is 0.8 mm. The head is scissor shaped, which is formed by two pieces of movable scissor edges. When the scissor edges are open, it is a miniature scissor. When they are closed, its appearance looks like type VI sword edge acupotome. The edge line and the handle are within the same plane. Only within the same plane can distinguish the direction of the edge line in the human body from the direction of the handle after the needle inserts into the muscle.

Type IX model 2 acupotome has same structure as type IX model 1, wherein the body is 9 cm long. Type IX model 3 acupotome has same structure as type IX model 1, wherein the body is 4 cm.

This acupotome is used for cutting and dissoluting nervous muscles and fascia, and dissecting small tumor in the body.

(10) Type X Revolving Edge Acupotome

According to the size, it has three models, i.e., type X model 1, type X model 2, and type X model 3.

Type X model 1 acupotome has a total length of 14.5 cm, in which the handle is 2 cm long, the body is 12 cm long, and the head is 0.5 cm long. The handle is flat trapezium shaped. The body is cylinder form with a diameter of 1.2 mm, which has a thin bore therein. The head has three-piece micro loose-leaf edges. When the loose-leaf edges are open, they look like fan leaf of an electric fan. When closed, the acupotome looks similar to type I acupotome. The head is wedge form, the end of which is flat with edge. The edge line is 1 mm, and the edge is flat neat. The edge line and the handle are within the same plane. Only within the same plane can distinguish the direction of the edge line in the human body from the direction of the handle after the needle inserts into the muscle.

Type X model 2 acupotome has same structure as type X model 1, wherein the body is 9 cm long. Type X model 3 acupotome has same structure as type X model 1, wherein the body is 4 cm long.

This acupotome is used for promoting blood circulation and other tiny piping jam.

(11) Type XI Probe Form Acupotome

According to the size, it has three models, i.e., type XI model 1, type XI model 2, and type XI model 3.

Type XI model 1 acupotome has a total length of 15 cm, in which the handle is 3 cm, the body is 10 cm with one side 7 having an edge, and the head is 2 cm. Its front view and side view both look alike a probe head. In the front view, the tip forms a 45° degree angle. The angle and the body with edge side have edges and the other side does not have edge. Both sides and the body lines are connected in smooth curve. In the side view, the head is wedge form. The handle is flat calabash shaped. The body is flat slip form, with a width of 2 mm, a thickness of 0.8 mm at one side, and the side 7 is edge. The edge and the handle are within the same plane. Only within the same plane can distinguish the direction of the edge line in the body from the direction of the handle after the needle inserts into the muscle.

Type XI model 2 acupotome has same structure as type XI model 1, wherein the body is 7 cm long. Type XI model 3 acupotome has same structure as type XI model 1, wherein the body is 5 cm long.

This acupotome is used for extirpating nodular bodies and other lesion.

(12) Type XII Curved Form Acupotome

According to the size, it has three models, i.e., type XII model 1, type XII model 2, and type XII model 3.

Type XII model 1 acupotome has a total length of 15 cm, in which the handle is 3 cm, the body is 10 cm with one side 8 having an edge, and the head is 2 cm. The head is circular cone shaped. The handle is flat calabash shaped. The body has a cross section of 45° degree angle sector, with an edge at the angle; the other side has a thickness of 0.8 mm. The end bends 180°. The bending area to the head side has an edge and connects to the head. The head tip faces the handle, with a bore therein.

Type XII model 2 acupotome has same structure as type XII model 1, wherein the body is 7 cm long. Type XII model 3 acupotome has same structure as type XII model 1, wherein the body is 5 cm long.

This acupotome is used for extirpating nodular bodies and other lesion.

This series of closed operation acupotome include type I flat neat acupotome, type II osteotome acupotome, type III oblique neat acupotome, type IV circular edge acupotome, type V concave edge acupotome, type VI sword edge acupotome, type VII injection acupotome, type VIII bill edge acupotome, type IX scissor edge acupotome, type X revolving edge acupotome, type XI probe form acupotome, and type XII curved form acupotome, all together 12 types with 33 models. Each type of acupotome has an acupotome handle, an acupotome body and an acupotome head, connecting in that order. The features lie on that different types of acupotome have different shapes of handle, body or head; same type different models acupotome have different length or diameter of the acupotome body, to accommodate treatment for various diseases and various parts and depth of the same disease.

The handle of the above accupotome: The accupotome handle of type II, type VII and type X is in flat trapezium shape, the accupotome handle of other types is in flat calabash shape. The accupotome handle of type VII has a jack that connects to an injector, the accupotome handle of type IX has two wires that connects with scissor edge of the head, and the accupotome handle of type X has a wire that connects with three-piece micro loose-leaf blade of the head.

The body of the above accupotome: Type XI is in flat slip shape with a blade at one side, type XII has a cross section of 45° degree angle sector with a blade at the acute angle, and other types is column shaped in which type VII, 1× and X has a thin bore therein.

The head of the above accupotome: Type X is a three-piece micro loose-leaf blade, type XI is probe form, type XII is coniform, and other types is wedge form with various shaped blade at the tips.

The flat surface of the handle and the flat wedge surface of the head are in the same plane, with one direction.

The handle of the above accupotome is made of 2Col3 material, and the body and head are made of 4Col3 material.

We claim:

1. A series of closed operation accupotome, including 12 types with 33 models, the 12 types of accupotome are type I, type II, type III, type IV, type V, type VI, type VII, type VIII, type IX, type X, type XI, and type XII, respectively, each type of accupotome having an accupotome handle, an accupotome body and an accupotome head, connecting in that order; wherein different type of accupotome having different shape of the accupotome handle, the accupotome body or the accupotome head, same type but different model accupotome having different length or diameter of the accupotome body, so as to accommodate treatment for various diseases, and various parts and depth of same disease.

2. The series of closed operation accupotome according to claim 1, wherein the accupotome handle of type II, type VII and type X accupotome is in flat trapezium shape, the accupotome handle of other types is in flat calabash shape, the accupotome handle of type VIII having a jack to connect to an injector, the accupotome handle of type IX having two wire to connect with scissor blade of the head, and the accupotome handle of type X having a wire to connect with three-piece micro loose-leaf blade of the head.

3. The series of closed operation accupotome according to claim 1, wherein the accupotome body of type XI accupotome is in flat strip shape with a blade at one side, the accupotome body of type XII having a cross section of 45′-angle sector with a blade at the acute angle, and the accupotome body of other types is in column shape, wherein the accupotome body of type VII, IX and X having a thin bore.

4. The series of closed operation accupotome according to claim 1, wherein the accupotome head of type X accupotome is a three-piece micro loose-leaf blade, the accupotome head of type XI is probe form, the accupotome head of type XII is coniform, and the accupotome head of other types is wedged form with various shaped tips.

5. The series of closed operation accupotome according to claim 1, wherein the accupotome handle has a flat surface, the accupotome head has a flat wedged surface, and both surfaces are in the same plane.

6. The series of closed operation accupotome according to claim 1, wherein the accupotome handle is made of 2Col3 material, and the accupotome body and head are made of 4Col3 material.
